(19) United States
(12) Patent Application Publication
(10) Pub. No.: US 2019/0321238 A1
(43) Pub. Date: Oct. 24, 2019

(54) SMART DIAPER SENSOR AND SENSOR CONTROL METHOD THEREOF

(71) Applicant: TamKang University, New Taipei City (TW)

(72) Inventors: HORNG-YUAN SHIH, Taipei City (TW); JEN-SHIUN CHIANG, Taoyuan City (TW); WEI-BIN YANG, Hsinchu City (TW); CHI-HSIUNG WANG, Yunlin County (TW); YU-CHUAN CHANG, New Taipei City (TW); CHENG-WEI YANG, Taipei City (TW)

(21) Appl. No.: 16/150,767

(22) Filed: Oct. 3, 2018

(30) Foreign Application Priority Data
Apr. 18, 2018 (TW) 107113237

(51) Int. Cl. A61F 13/42; A61B 5/00; A61F 13/49

(52) U.S. Cl. CPC A61F 13/42; A61F 2013/424; A61F 13/49; A61B 5/6808

(57) ABSTRACT

A smart diaper sensor, which may be disposed inside a diaper, and may include a sensing element and a wireless RF circuit. The wireless RF circuit may include a transmitter and a wake-up receiver. The wake-up receiver may receive a wake-up signal transmitted from a backend platform to wake up the transmitter. The sensing element may sense the environmental status inside the diaper to generate a sensing signal. The transmitter may receive the sensing signal, and then transmit the sensing signal to the backend platform.

### CROSS REFERENCE TO RELATED APPLICATION

This application also claims priority to Taiwan Patent Application No. 107113237 filed in the Taiwan Patent Office on Apr. 18, 2018, the entire content of which is incorporated herein by reference.

### TECHNICAL FIELD

The present disclosure is related to a sensor, in particular to a smart diaper sensor. The present disclosure further related to the sensor control method of the smart diaper sensor.

### BACKGROUND

With the accelerating of aging society, old age population keeps increasing. Many disabled elderly persons or long-term bedridden patients need to wear diapers; however, if a patient wearing a diaper makes a bowel movement but the diaper fails to be changed in a short time, the patient may suffer from eczema, urinary tract infection or other diseases, which may significantly influence the health of the patient and reduce the working efficiency of the nurses.

Accordingly, various electronic diapers are developed so as to solve the above problem. However, as the sizes of these currently available electronic diapers are large, the patients wearing the diapers may feel uncomfortable.

Moreover, these currently available electronic diapers should be powered by batteries, so the weights of these diapers increase accordingly; besides, the capacities of batteries are limited, so the electronic diapers cannot be continuously used for a long time, which is very inconvenient in use.

Furthermore, these currently available electronic diapers tend to malfunction, so may not correctly detect whether the patients make a bowel movement, which tends to result in negative influences to the patients.

Therefore, it has become an important issue to provide a smart diaper capable of solving the various problems of the currently available electronic diapers.

### SUMMARY

The present disclosure is related to a smart diaper sensor, which may be disposed inside a diaper. In an embodiment of the disclosure, the smart diaper sensor may include a sensing element and a wireless RF circuit. The wireless RF circuit may include a transmitter and a wake-up receiver. The wake-up receiver may receive a wake-up signal transmitted from a backend platform to wake up the transmitter. The sensing element may sense the environmental status inside the diaper to generate a sensing signal. The transmitter may receive the sensing signal, and then transmit the sensing signal to the backend platform.

In an embodiment of the disclosure, the smart diaper sensor may further include an analog front-end circuit coupled to the sensing element.

In an embodiment of the disclosure, the smart diaper sensor may further include an analog-to-digital converter coupled to the analog front-end circuit and the wireless RF circuit.

In an embodiment of the disclosure, the analog front-end circuit may receive the sensing signal and pre-process the sensing signal to generate a pre-processed signal.

In an embodiment of the disclosure, the analog-to-digital converter may receive the pre-processed signal, convert the pre-processed signal into a digital signal and transmit the digital signal to the transmitter; the transmitter may transmit the digital signal to the backend platform.

In an embodiment of the disclosure, the smart diaper sensor may further include an energy harvester coupled to the wireless RF circuit, the analog front-end circuit and the analog-to-digital converter; the energy harvester may harvest the environment energy to power the wireless RF circuit, the analog front-end circuit and the analog-to-digital converter.

In an embodiment of the disclosure, the wake-up receiver may wake up the wireless RF circuit, the analog front-end circuit and the analog-to-digital converter after receiving the wake-up signal transmitted from the backend platform, and the energy harvester may power the wireless RF circuit, the analog front-end circuit and the analog-to-digital converter.

In an embodiment of the disclosure, the environmental status may include one or more than one of humidity, smell and temperature.

In an embodiment of the disclosure, the wake-up receiver may periodically receive the wake-up signal from the backend platform.

In an embodiment of the disclosure, the wireless RF circuit may further include an activation test circuit coupled to the wake-up circuit; the activation test circuit may execute a test process to test whether the sensing element and the transmitter work normally.

The present disclosure is related to a sensor control method. In an embodiment of the disclosure, the sensor control method may include the following steps: receiving a wake-up signal transmitted from a backend platform by a wake-up receiver of a wireless RF circuit so as to wake up a transmitter of the wireless RF circuit; sensing an environmental status inside a diaper by a sensing element to generate a sensing signal; and receiving the sensing signal and transmitting the sensing signal to a backend platform by the transmitter of the wireless RF circuit.

In an embodiment of the disclosure, the step of sensing the environmental status inside the diaper by the sensing element to generate the sensing signal may further include the following step: receiving and pre-processing the sensing signal by an analog front-end circuit to generate a pre-processed signal.

In an embodiment of the disclosure, the step of sensing the environmental status inside the diaper by the sensing element to generate the sensing signal may further include the following step: receiving the pre-processed signal and converting the pre-processed signal into a digital signal and transmitting the digital signal to the transmitter by an analog-to-digital converter; and transmitting the digital signal to the backend platform by the transmitter.

In an embodiment of the disclosure, the sensor control method may further include the following step: harvesting an environment energy by an energy harvester to power the wireless RF circuit, the analog front-end circuit and the analog-to-digital converter.

In an embodiment of the disclosure, the step of receiving the wake-up signal transmitted from the backend platform by the wake-up receiver of the wireless RF circuit so as to wake up the transmitter of the wireless RF circuit may further include the following step: waking up the analog front-end circuit and the analog-to-digital converter by the wake-up receiver.

In an embodiment of the disclosure, the environmental status may include one or more than one of humidity, smell and temperature.

In an embodiment of the disclosure, the wake-up receiver may periodically receive the wake-up signal from the backend platform.

In an embodiment of the disclosure, the sensor control method may further include the following step: executing a test process by an activation test circuit of the wireless RF circuit so as to test whether the sensing element and the transmitter of the wireless RF circuit work normally.

The smart diaper sensor and the sensor control method thereof according to the embodiments of the present disclosure may have the following advantages:

(1) According to one embodiment of the present disclosure, the smart diaper sensor can be disposed in the diaper and can effectively detect whether the user makes a bowel movement; besides, the smart diaper sensor can transmit a warning signal to the backend platform to remind the nurse to change the diaper on time, which can effectively the negative influences caused by the diaper and can increase the working efficiency of the nurses.

(2) According to one embodiment of the present disclosure, the smart diaper sensor can include an activation test circuit, which can execute a test process to test whether the smart diaper sensor works normally in order to avoid that the smart diaper malfunctions, which can further prevent from the negative influences caused by the diaper.

(3) According to one embodiment of the present disclosure, the size of the smart diaper sensor can be minimized, so the patient will not feel uncomfortable even if having worn the diaper for a long time.

(4) According to one embodiment of the present disclosure, the smart diaper sensor can include an energy harvester able to harvest energy in order to power the smart diaper sensor, so the smart diaper sensor can work for a long time without batteries, which is very convenient in use.

(5) According to one embodiment of the present disclosure, the smart diaper sensor can be powered by the electricity provided by an energy harvester, so the smart diaper sensor can work for a long time without batteries, which can significantly reduce cost and decrease environmental pollution.

Further scope of applicability of the present application will become more apparent from the detailed description given hereinafter. However, it should be understood that the detailed description and specific examples, while indicating exemplary embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The present disclosure will become more fully understood from the detailed description given herein below and the accompanying drawings which are given by way of illustration only, and thus are not limitative of the present disclosure and wherein:

FIG. 1 is a circuit diagram of a smart diaper sensor in accordance with a first embodiment of the present disclosure.

FIG. 2 is a flow chart of the first embodiment.

FIG. 3 is a circuit diagram of a smart diaper sensor in accordance with a second embodiment of the present disclosure.

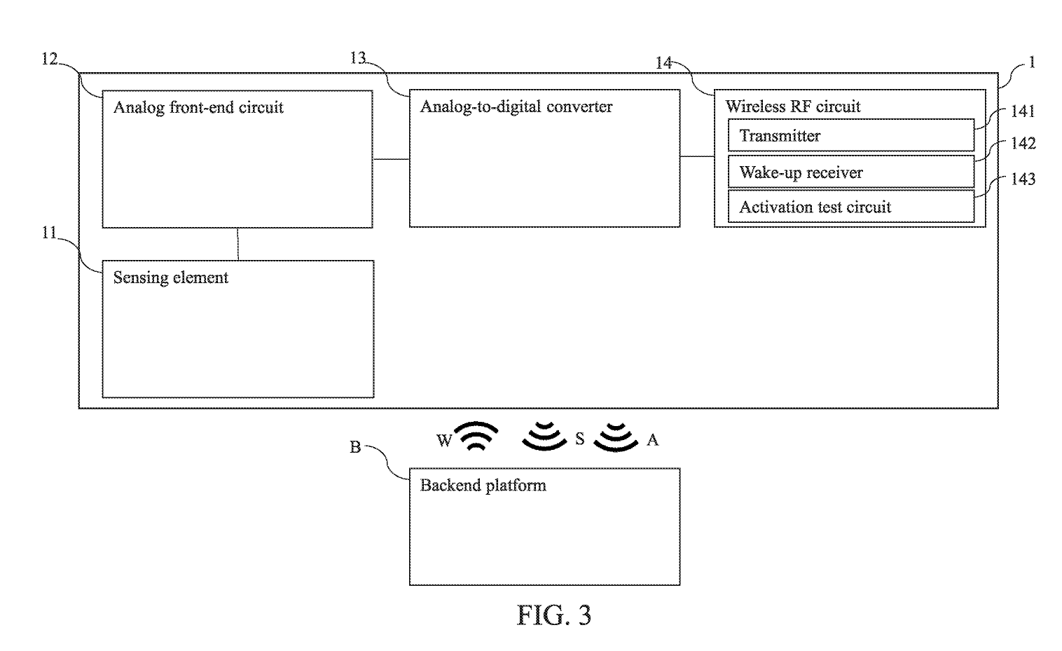

FIG. 4 is a flow chart of the second embodiment.

FIG. 5 is a circuit diagram of a smart diaper sensor in accordance with a third embodiment of the present disclosure.

FIG. 6 is a flow chart of the third embodiment.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing. It should be understood that, when it is described that an element is “coupled” or “connected” to another element, the element may be “directly coupled” or “directly connected” to the other element or “coupled” or “connected” to the other element through a third element. In contrast, it should be understood that, when it is described that an element is “directly coupled” or “directly connected” to another element, there are no intervening elements.

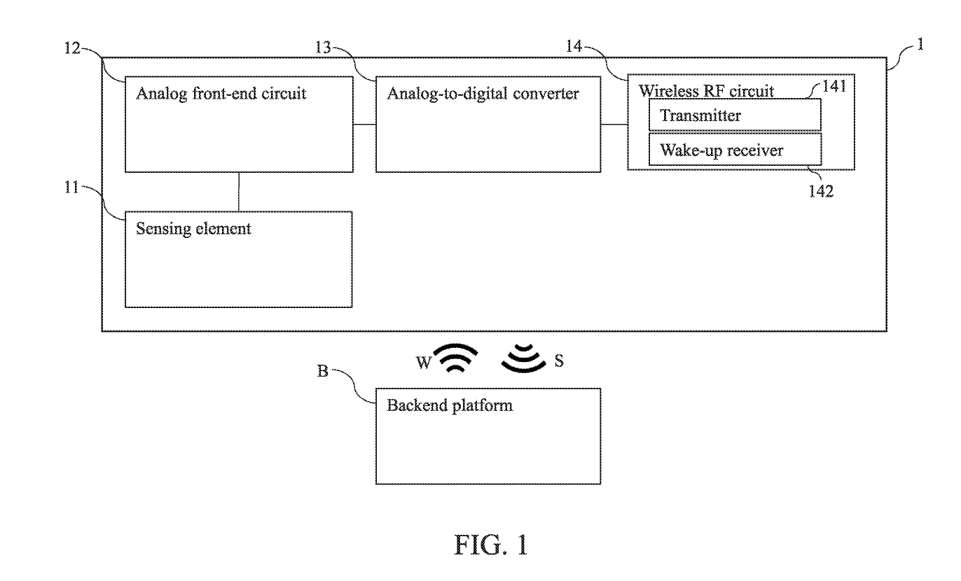

Please refer to FIG. 1, which is a circuit diagram of a smart diaper sensor in accordance with a first embodiment of the present disclosure. The smart diaper sensor 1 can be disposed inside a diaper to detect whether the patient wearing the diaper makes a bowel movement. As shown in FIG. 1, the smart diaper sensor may include a sensing element 11, an analog front-end circuit 12, an analog-to-digital converter 13 and a wireless RF circuit 14.

The wireless RF circuit 14 is coupled to the analog-to-digital converter 13, and includes a transmitter 141 and a wake-up receiver 142.

The analog front-end circuit 12 is coupled to the analog-to-digital converter 13.

The sensing element 11 is coupled to the analog-to-digital converter 13.

The sensing element 11 is coupled to the analog front-end circuit 12; in the embodiment, the sensing element 11 may be a passive component.

Next, the backend platform can periodically transmit a wake-up signal W to the wake-up receiver 142, and then the wake-up receiver 142 wakes up the analog front-end circuit 12, the analog-to-digital converter 13 and the receiver 141. Afterward, the sensing element 11 senses the environmental status inside the diaper to generate a sensing signal S. In an embodiment, the environmental status may include one or more than one of the humidity, the smell and the temperature of the diaper.

Then, the analog front-end circuit 12 receives the sensing signal S and pre-processes the sensing signal S to generate a pre-processed signal, and the analog-to-digital converter 13 receives the pre-processed signal, converts the pre-processed signal into a digital signal and transmit the digital signal to the transmitter 141. Finally, the transmitter 141 transmits the digital signal to the backend platform B.

Thus, after the patient makes a bowel movement, the nurse can be immediately notified that it is time to change the diaper of the patient, so can effectively avoid that the patient suffers from eczema, urinary tract infection or other diseases to prevent from negative influences to the health of the patient and can significantly better the working efficiency of the nurse; in an embodiment, the backend platform B may be various electronic devices, such as computer, server or smart phone.

In addition, the above circuit design can minimize the size of the smart diaper sensor 1, so the patient will not feel uncomfortable even if wearing the diaper for a long time.

The embodiment just exemplifies the present disclosure and is not intended to limit the scope of the present disclosure; any equivalent modification and variation according to the spirit of the present disclosure is to be also included within the scope of the following claims and their equivalents.

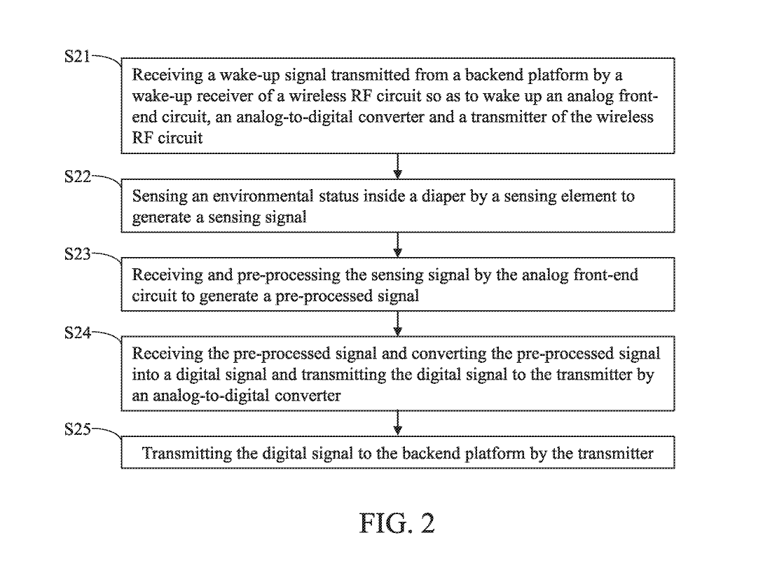

Please refer to FIG. 2, which is a flow chart of the first embodiment. The sensor control method of the smart diaper sensor 1 in the embodiment includes the following steps:

Step 21: receiving a wake-up signal transmitted from a backend platform by a wake-up receiver of a wireless RF circuit so as to wake up an analog front-end circuit, an analog-to-digital converter and a transmitter of the wireless RF circuit.

Step 22: sensing an environmental status inside a diaper by a sensing element to generate a sensing signal.

Step 23: receiving and pre-processing the sensing signal by the analog front-end circuit to generate a pre-processed signal.

Step 24: receiving the pre-processed signal and converting the pre-processed signal into a digital signal and transmitting the digital signal to the transmitter by an analog-to-digital converter.

Step 25: transmitting the digital signal to the backend platform by the transmitter.

Please refer to FIG. 2, which is a circuit diagram of a smart diaper sensor in accordance with a second embodiment of the present disclosure. The smart diaper sensor 1 can be disposed inside a diaper to detect whether the patient wearing the diaper makes a bowel movement. As shown in FIG. 2, the smart diaper sensor may include a sensing element 11, an analog front-end circuit 12, an analog-to-digital converter 13 and a wireless RF circuit 14.

The wireless RF circuit 14 is coupled to the analog-to-digital converter 13, and includes a transmitter 141 and a wake-up receiver 142.

The analog front-end circuit 12 is coupled to the analog-to-digital converter 13.

The sensing element 11 is coupled to the analog-to-digital converter 13.

The sensing element 11 is coupled to the analog front-end circuit 12.

The above elements are similar to those of the previous embodiment, so will not be described in herein. The difference between the embodiment and the previous embodiment is that the wireless RF circuit 14 further includes an activation test circuit 143. The activation test circuit 143 is coupled to the wake-up circuit 142 and executes a test process to test whether the sensing element 11, the analog front-end circuit 12, the analog-to-digital converter 13 and the transmitter 141 work normally. If any one of the above elements cannot work normally, the activation test circuit 143 generates a warning signal A and transmits the warning signal A to the backend platform B so as to make sure that the smart diaper sensor 1 can work normally.

The embodiment just exemplifies the present disclosure and is not intended to limit the scope of the present disclosure; any equivalent modification and variation according to the spirit of the present disclosure is to be also included within the scope of the following claims and their equivalents.

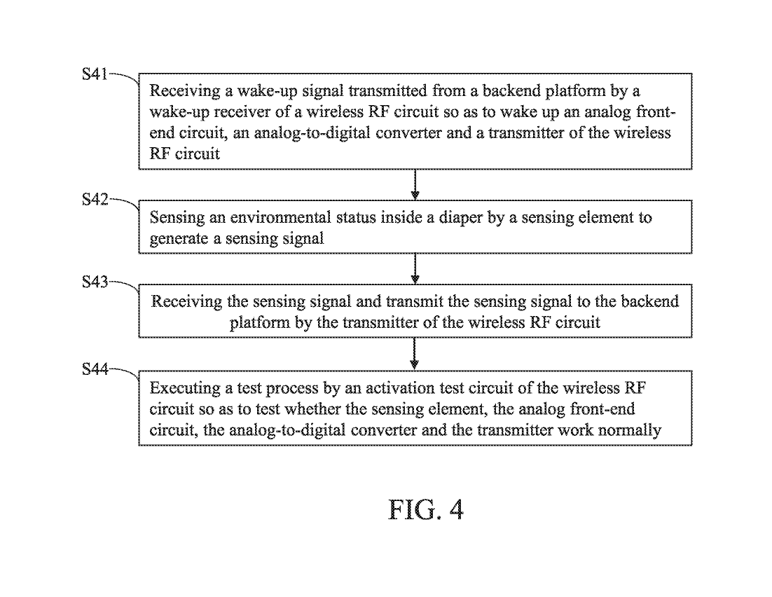

Please refer to FIG. 4, which is a flow chart of the second embodiment. The sensor control method of the smart diaper sensor 1 in the embodiment includes the following steps:

Step 41: receiving a wake-up signal transmitted from a backend platform by a wake-up receiver of a wireless RF circuit so as to wake up an analog front-end circuit, an analog-to-digital converter and a transmitter of the wireless RF circuit.

Step 42: sensing an environmental status inside a diaper by a sensing element to generate a sensing signal.

Step 43: receiving the sensing signal and transmit the sensing signal to the backend platform by the transmitter of the wireless RF circuit.

Step 44: executing a test process by an activation test circuit of the wireless RF circuit so as to test whether the sensing element, the analog front-end circuit, the analog-to-digital converter and the transmitter work normally.

It is worthy to point out that if a patient wearing a diaper makes a bowel movement but the diaper fails to be changed in a short time, the patient may suffer from eczema, urinary tract infection or other diseases, which may significantly influence the health of the patient and reduce the working efficiency of the nurses. On the contrary, according to one embodiment of the present disclosure, the smart diaper sensor can be disposed in the diaper and can effectively detect whether the user makes a bowel movement; besides, the smart diaper sensor can transmit a warning signal to the backend platform to remind the nurse to change the diaper on time, which can effectively the negative influences caused by the diaper and can increase the working efficiency of the nurses.

Moreover, the currently available electronic diapers tend to malfunction, so may not correctly detect whether the patients make a bowel movement, which tends to result in negative influences to the patients. On the contrary, according to one embodiment of the present disclosure, the smart diaper sensor can include an activation test circuit, which can execute a test process to test whether the smart diaper sensor works normally in order to avoid that the smart diaper malfunctions, which can further prevent from the negative influences caused by the diaper.

Furthermore, as the sizes of the currently available electronic diapers are large, the patients wearing the diapers may feel uncomfortable. On the contrary, according to one embodiment of the present disclosure, the size of the smart diaper sensor can be minimized, so the patient will not feel uncomfortable even if having worn the diaper for a long time.

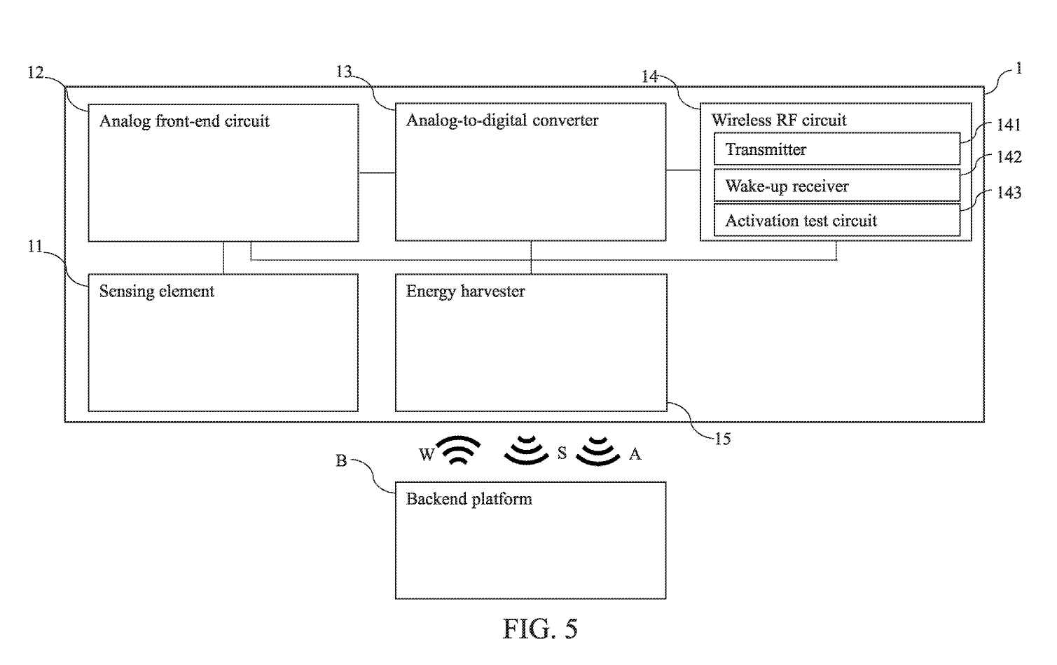

Please refer to FIG. 5, which is a circuit diagram of a smart diaper sensor in accordance with a third embodiment of the present disclosure. The smart diaper sensor 1 can be disposed inside a diaper to detect whether the patient wearing the diaper makes a bowel movement. As shown in FIG. 5, the smart diaper sensor may include a sensing element 11, an analog front-end circuit 12, an analog-to-digital converter 13 and a wireless RF circuit 14.

The wireless RF circuit 14 is coupled to the analog-to-digital converter 13, and includes a transmitter 141, a wake-up receiver 142 and an activation test circuit 143.

The analog front-end circuit 12 is coupled to the analog-to-digital converter 13.

The sensing element 11 is coupled to the analog-to-digital converter 13.

The sensing element 11 is coupled to the analog front-end circuit 12.

The above elements are similar to those of the previous embodiment, so will not be described in herein. The difference between the embodiment and the previous embodiment is that the smart diaper sensor 1 further includes an energy harvester 15. The energy harvester 15 is coupled to the wireless RF circuit 14, the analog front-end circuit 12 and the analog-to-digital converter 13, and harvests the environmental energy so as to power the wireless RF circuit 14, the analog front-end circuit 12 and the analog-to-digital converter 13; in an embodiment, the energy harvest 15 harvests one or more than one of light energy, thermal energy, vibration energy and electromagnetic energy so as to power the wireless RF circuit 14. After receiving the wake-up signal W from the backend platform B, the wake-up receiver 142 wakes up the wireless RF circuit 14, the analog front-end circuit 12 ad the analog-to-digital converter 13; then, the energy harvester 15 powers the wireless RF circuit 14, the analog front-end circuit 12 and the analog-to-digital converter 13.

As described above, the smart diaper sensor 1 of the embodiment can be powered by the environmental energy harvested by the energy harvester 15, so the smart diaper 1 sensor can work for a long time without batteries, which is very convenient in use and can significantly reduce cost and decrease environmental pollution.

The embodiment just exemplifies the present disclosure and is not intended to limit the scope of the present disclosure; any equivalent modification and variation according to the spirit of the present disclosure is to be also included within the scope of the following claims and their equivalents.

It is worthy to point out that the currently available electronic diapers should be powered by batteries, so the weights of these diapers increase accordingly; besides, the capacities of batteries are limited, so the electronic diapers cannot be continuously used for a long time, which is very inconvenient in use. On the contrary, according to one embodiment of the present disclosure, the smart diaper sensor can include an energy harvester able to harvest energy in order to power the smart diaper sensor, so the smart diaper sensor can work for a long time without batteries, which is very convenient in use.

Besides, according to one embodiment of the present disclosure, the smart diaper sensor can be powered by the electricity provided by the energy harvester, so the smart diaper sensor can work for a long time without batteries, which can significantly reduce cost and decrease environmental pollution. Thus, the smart diaper sensor according to the embodiments of the present discloser definitely has an inventive step.

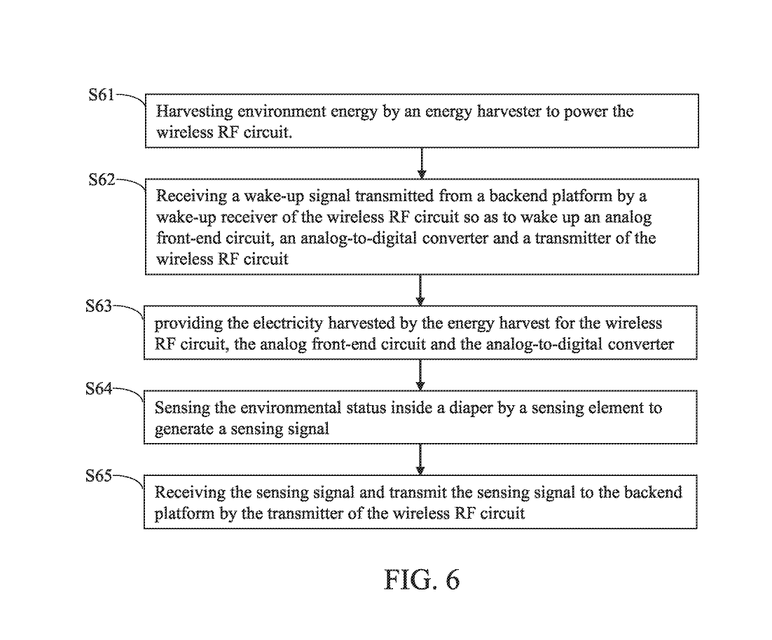

Please refer to FIG. 6, which is a flow chart of the third embodiment. The sensor control method of the smart diaper sensor 1 in the embodiment includes the following steps:

Step 61: harvesting environment energy by an energy harvester to power the wireless RF circuit.

Step 62: receiving a wake-up signal transmitted from a backend platform by a wake-up receiver of the wireless RF circuit so as to wake up an analog front-end circuit, an analog-to-digital converter and a transmitter of the wireless RF circuit.

Step 63: providing the electricity harvested by the energy harvest for the wireless RF circuit, the analog front-end circuit and the analog-to-digital converter.

Step 64: sensing the environmental status inside a diaper by a sensing element to generate a sensing signal.

Step 65: receiving the sensing signal and transmit the sensing signal to the backend platform by the transmitter of the wireless RF circuit.

To sum up, according to one embodiment of the present disclosure, the smart diaper sensor can be disposed in the diaper and can effectively detect whether the user makes a bowel movement; besides, the smart diaper sensor can transmit a warning signal to the backend platform to remind the nurse to change the diaper on time, which can effectively the negative influences caused by the diaper and can increase the working efficiency of the nurses.

Also, according to one embodiment of the present disclosure, the smart diaper sensor can include an activation test circuit, which can execute a test process to test whether the smart diaper sensor works normally in order to avoid that the smart diaper malfunctions, which can further prevent from the negative influences caused by the diaper.

Besides, according to one embodiment of the present disclosure, the size of the smart diaper sensor can be minimized, so the patient will not feel uncomfortable even if having worn the diaper for a long time.

Moreover, according to one embodiment of the present disclosure, the smart diaper sensor can include an energy harvester able to harvest energy in order to power the smart diaper sensor, so the smart diaper sensor can work for a long time without batteries, which is very convenient in use.

Furthermore, according to one embodiment of the present disclosure, the smart diaper sensor can be powered by the electricity provided by an energy harvester, so the smart diaper sensor can work for a long time without batteries, which can significantly reduce cost and decrease environmental pollution.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A smart diaper sensor applicable to a diaper, comprising:
a sensing element; and
a wireless RF circuit, comprising a transmitter and a wake-up receiver;
wherein the wake-up receiver receives a wake-up signal transmitted from a backend platform to wake up the transmitter; the sensing element senses an environmental status inside the diaper to generate a sensing signal; the transmitter receives the sensing signal and transmit the sensing signal to the backend platform.

2. The smart diaper sensor of claim 1, further comprising an analog front-end circuit coupled to the sensing element.

3. The smart diaper sensor of claim 2, further comprising an analog-to-digital converter coupled to the analog front-end circuit and the wireless RF circuit.

4. The smart diaper sensor of claim 3, wherein the analog front-end circuit receives the sensing signal and pre-processes the sensing signal to generate a pre-processed signal.

5. The smart diaper sensor of claim 4, wherein the analog-to-digital converter receives the pre-processed signal, converts the pre-processed signal into a digital signal and transmits the digital signal to the transmitter; the transmitter transmits the digital signal to the backend platform.

6. The smart diaper sensor of claim 3, further comprising an energy harvester coupled to the wireless RF circuit, the analog front-end circuit and the analog-to-digital converter, wherein the energy harvester harvests an environment energy to power the wireless RF circuit, the analog front-end circuit and the analog-to-digital converter.

7. The smart diaper sensor of claim 6, wherein the wake-up receiver wakes up the wireless RF circuit, the analog front-end circuit and the analog-to-digital converter after receiving the wake-up signal transmitted from the backend platform, and the energy harvester powers the wireless RF circuit, the analog front-end circuit and the analog-to-digital converter.

8. The smart diaper sensor of claim 1, wherein the environmental status comprises one or more than one of a humidity, smell and a temperature.

9. The smart diaper sensor of claim 1, wherein the wake-up receiver periodically receives the wake-up signal from the backend platform.

10. The smart diaper sensor of claim 1, wherein the wireless RF circuit further comprises an activation test circuit coupled to the wake-up circuit; the activation test circuit executes a test process to test whether the sensing element and the transmitter work normally.

11. A sensor control method, comprising:
receiving a wake-up signal transmitted from a backend platform by a wake-up receiver of a wireless RF circuit so as to wake up a transmitter of the wireless RF circuit;
sensing an environmental status inside a diaper by a sensing element to generate a sensing signal; and
receiving the sensing signal and transmitting the sensing signal to a backend platform by the transmitter of the wireless RF circuit.

12. The sensor control method of claim 11, wherein a step of sensing the environmental status inside the diaper by the sensing element to generate the sensing signal further comprises:
receiving and pre-processing the sensing signal by an analog front-end circuit to generate a pre-processed signal.

13. The sensor control method of claim 12, wherein the step of sensing the environmental status inside the diaper by the sensing element to generate the sensing signal further comprises:
receiving the pre-processed signal and converting the pre-processed signal into a digital signal and transmitting the digital signal to the transmitter by an analog-to-digital converter; and
transmitting the digital signal to the backend platform by the transmitter.

14. The sensor control method of claim 13, further comprising:
harvesting an environment energy by an energy harvester to power the wireless RF circuit, the analog front-end circuit and the analog-to-digital converter.

15. The sensor control method of claim 14, wherein a step of receiving the wake-up signal transmitted from the backend platform by the wake-up receiver of the wireless RF circuit so as to wake up the transmitter of the wireless RF circuit further comprises:
waking up the analog front-end circuit and the analog-to-digital converter by the wake-up receiver.

16. The sensor control method of claim 11, wherein the environmental status comprises one or more than one of a humidity, smell and a temperature.

17. The sensor control method of claim 11, wherein the wake-up receiver periodically receives the wake-up signal from the backend platform.

18. The sensor control method of claim 11, further comprising:
executing a test process by an activation test circuit of the wireless RF circuit so as to test whether the sensing element and the transmitter of the wireless RF circuit work normally.
